(19) United States
(12) Patent Application Publication
(10) Pub. No.: US 2024/0141407 A1
(43) Pub. Date: May 2, 2024

(54) METHOD FOR TESTING RECENTNESS OF INSECT FECAL PELLETS AND ASSOCIATED KITS

(71) Applicant: The Regents of the University of California, Oakland, CA (US)

(72) Inventors: Dong-Hwan Choe, Riverside, CA (US); Kathleen Campbell, Riverside, CA (US); Michael K. Rust, Riverside, CA (US)

(73) Assignee: The Regents of the University of California, Oakland, CA (US)

(21) Appl. No.: 18/384,338

(22) Filed: Oct. 26, 2023

Related U.S. Application Data
(60) Provisional application No. 63/419,433, filed on Oct. 26, 2022.

(51) Int. Cl. C12Q 1/30

(52) U.S. Cl. CPC C12Q 1/30; G01N 2333/43552

(57) ABSTRACT

Described herein are methods for characterizing the age of an insect fecal pellet, where the method can comprise: (1) obtaining insect fecal pellet material to be tested; (2) exposing the pellet material to an H₂O₂ solution; and (3) detecting the presence of generated oxygen, where the amount of oxygen generated is inversely proportional to the age of the insect pellet. Also described are kits for carrying out the method.

### CROSS-REFERENCE TO RELATED APPLICATIONS

This application claims the benefit of a U.S. Provisional patent application No. 63/419,433 titled “METHOD FOR TESTING RECENTNESS OF INSECT FECAL PELLETS AND ASSOCIATED KITS” and dated Oct. 26, 2022, the contents of which are incorporated herein in their entirety.

This disclosure claims the benefit of the filing dates of U.S. Provisional Patent Application No. 63/419,433, filed Oct. 26, 2022, the entire contents of all of which are hereby expressly incorporated by reference.

### FIELD OF INVENTION

Described is a method for testing the recentness of insect fecal pellets, such as termite pellets, and associated kits for implementing the method.

### BACKGROUND

Drywood termites are cryptic insects and their colonies are difficult to detect. They live inside a piece of wood; and except during periods when their winged reproductives swarm or when repair work is being done on infested structures, they are seldom seen. Sampling actual termites from the wood often requires extensive/invasive investigation. During a structural inspection for drywood termites, inspectors look for feeding damage, shed wings, fecal pellets, and kickout holes, i.e. small holes (less than 2 mm in diameter) through which termites push fecal pellets out of the wood. In fact, in structures infested by drywood termites, it is common to find fecal pellets. These fecal pellets have six hexagonal sides and this shape is diagnostic for drywood termites. However, it is not possible to determine, from the appearance of fecal pellets alone, whether the infestation is currently active. In other words, when fecal pellets appear after remedial treatment of a structure (e.g., heat treatment or fumigation), it is difficult to determine whether this indicates that termites in the structure are still alive and active or not. This is due to the lack of reliable methods to determine the age of the fecal pellets—new or old. If there is a simple and reliable method to determine if the fecal pellets are recently produced (i.e., produced by the termites within a few months or a year), it will make a highly useful tool for structural termite pest control and inspection.

It has been investigated if cuticular hydrocarbon profiles in the drywood termite fecal pellets could be used to determine the age of fecal pellets. See Lewis, et al. Quantitative changes in hydrocarbons over time in fecal pellets of Incisitermes minor may predict whether colonies are alive or dead 36 (11) J. Chemical Ecology 1199-206 (2010). In the Lewis study, termite fecal pellets were aged for periods of 0, 30, 90, and 365 days after collection. The cuticular hydrocarbon profiles obtained from the fecal pellets were qualitatively similar across all time periods. However, the relative quantities of certain individual hydrocarbons changed over time, with 19 of the 73 hydrocarbon peaks increasing or decreasing. While the study provided some intriguing information the hydrocarbon analyses involved extraction and clean up processes that require laboratory work as well as an organic solvent (e.g., hexane) making it is difficult to use by the general public. In addition, the analyses of the cuticular hydrocarbon required GC-MS (gas chromatography mass spectrometry) and special computer programs, which are not readily available for most of the pest management professionals and homeowners.

As a result, there is a need for a field usable, inexpensive diagnostic method and system that can detect the age of termite pellets so that an infestation can be determined as currently active.

### SUMMARY OF INVENTION

Some embodiments describe a method for characterizing the age of an insect fecal pellet, where the method can comprise: (1) obtaining insect fecal pellet material to be tested; (2) exposing the pellet material to an H₂O₂, or hydrogen peroxide, solution; and (3) detecting the presence of generated oxygen, where the amount of oxygen generated is inversely proportional to the age of the insect pellet. In some methods, the step of obtaining insect fecal pellet material can comprise obtaining at least about 0.001 g of fecal pellet material. In some embodiments, the step of obtaining insect fecal pellet material can comprise obtaining pellets excreted from a termite, a wood boring beetle, a carpenter ant, an ant, a cockroach, or a wood borer. For some embodiments, the step of obtaining insect fecal pellet material may comprise obtaining pellets excreted from a drywood termite. In some methods, the step of exposing the pellet material to an H₂O₂ solution can comprise exposing the pellet material to an aqueous solution of about 0.1 vol. % to about 10 vol. % H₂O₂. For some methods, the step of exposing the pellet material to an H₂O₂ solution can comprise placing the pellet material in physical communication with the H₂O₂ solution. In some method embodiments, the step of exposing can comprise applying the solution to the pellet material, immersing the pellet material, or submerging the pellet material. With some methods, the step of detecting the presence of generated oxygen can be by detecting bubbles. For some methods, the step of detecting the presence of generated oxygen can be by detecting floating pellets. Some methods can have the step of detecting the presence of generated oxygen by detecting a colorimetric reaction, where the H₂O₂ solution additionally comprises colorimetric oxygen sensor compounds or dyes.

Some embodiments describe a kit for detecting the age of an insect fecal pellet, where the kit can comprise: one or more reaction cartridges, where the reaction cartridges can comprise a plenum for containing at least 50 μL of solution comprising a H₂O₂ solution and the cartridge defines an orifice at the top of the cartridge for the addition of pellet material; a magnifying device; and a set of instructions, the instructions comprising the steps of: (1) obtaining insect pellet material to be tested, (2) exposing the pellet material to an H₂O₂ solution, and (3) detecting the presence of generated oxygen. In some kit embodiments, the reaction cartridges can be at least translucent so that light, natural or generated, can illuminate the H₂O₂ solution. For some kits, the reaction cartridges can further comprise a removable seal for sealing the cartridge orifice, which can be removed to expose the plenum for addition and examination of the samples. In some kits, the reaction cartridge can further comprise colorimetric oxygen sensor compounds or dyes mixed in with the H₂O₂ solution. For some kit embodiments, the magnification device can comprise a lens, a lens holder can define a cavity in which the lens can be physically secured by the lens holder within the cavity, a base for holding the reaction cartridge, where the base can define a measurement cavity, the measurement cavity extending from the upper portion of the base to where the reaction cartridge can be held, such that the reaction cartridge plenum and orifice, when inserted in the base all can be in optical communication with the lens, and a linkage for holding the lens holder a desired distance from the reaction cartridge. In some kits, the linkage can comprise a track whereby the attachment points of the lens holder can slide along the length of the linkage within the track such that the distance of the lens from the reaction cartridge can be adjusted. Some kits can further comprise a light source for illuminating the reaction cartridge plenum. Some kits can also further comprise one or more tools for transferring the fecal pellets for testing selected from the group consisting a small scoop, forceps, or an aspirator. In some kits, the instruction step of obtaining insect fecal pellet material to be tested can comprise the step of obtaining pellets excreted from a termite, a wood boring beetle, a carpenter ant, an ant, a cockroach, or a wood borer. For some kits, the instruction step of obtaining insect fecal pellet material to be tested can comprise the step of obtaining pellets excreted from a drywood termite.

Some embodiments, can describe another type of kit for detecting the age of an insect fecal pellet, where the kit can comprise: one or more reaction tubes, the reaction tube having a capacity of 500 μL to 50 mL and a closed end at the bottom, defining an orifice at the top; a removable cap, where the tube orifice can be sealed by the cap; an H₂O₂ solution, where a fraction of the internal volume of the one or more tubes is filled with the solution; and a set of instructions, the instructions comprising the steps of: (1) obtaining insect pellet material to be tested, (2) exposing the pellet material to an H₂O₂ solution, and (3) detecting the presence of generated oxygen. In some kits, the one or more reaction tubes can be individually stored in removable packaging to protect them from direct sunlight. With some kits, the reaction tube can further comprise colorimetric oxygen sensor compounds or dyes mixed in with the H₂O₂ solution. Some kits can further comprise one or more tools for transferring the fecal pellets for testing selected from the group consisting a small scoop, forceps, or an aspirator. In some kits, the instruction step of obtaining insect fecal pellet material to be tested can comprise the step of obtaining pellets excreted from a termite, a wood boring beetle, a carpenter ant, an ant, a cockroach, or a wood borer. For some kits, the instruction step of obtaining insect fecal pellet material to be tested can comprise the step of obtaining pellets excreted from a drywood termite.

These and other embodiments are described in greater detail below.

### BRIEF DESCRIPTION OF THE DRAWINGS

FIG. 1 is a schematic showing one possible method for detecting the age of an insect fecal pellet using H₂O₂ and detecting for the presence of generated oxygen.

FIG. 2 is a diagram showing one possible kit embodiment for implementing the method for detecting the age of an insect fecal pellet using H₂O₂. The embodiment involves magnification of the plenum containing the H₂O₂ for detection of generated oxygen.

FIG. 3 is an illustration of a non-limiting kit embodiment, showing that the distance of the lens holder from the plenum containing the H₂O₂ solution can be adjusted.

FIG. 4 is a diagram of a non-limiting kit embodiment, further comprising a light source that illuminates the plenum from underneath. In this particular embodiment, the light source located underneath the base and is battery powered, although any form of electrical energy source is contemplated.

FIG. 5 is a diagram of a non-limiting kit embodiment, further comprising a light source that illuminates the plenum from above. In this particular embodiment, the light source located on the linkage and is powered by an electrical plug, although any form of electrical energy source is contemplated.

FIG. 6 is a diagram showing one possible kit embodiment for implementing the method for detecting the age of an insect fecal pellet using H₂O₂. The embodiment involves observation of a tube containing the H₂O₂ for detection of generated oxygen.

FIG. 7 is a plot showing experimental data for percentage of pellets reacting against a curve-fit relationship for determining number of days since collection, or a measure of age of the pellet.

### DETAILED DESCRIPTION

The present invention provides for a method of detecting the age of insect pellets, more specifically termite pellets, as well as kits for practicing the method.

### Method for Detecting the Age of Insect Excrement

Described is a method for determining if the sampled insect fecal pellets are recently produced by living insect colony. Some methods the insect can comprise termites (e.g., drywood termites, damp wood termites), wood boring beetles (e.g., bark beetles, powderpost beetles), carpenter ants, ants, cockroach, or wood borers. Some methods the insect comprises drywood termites.

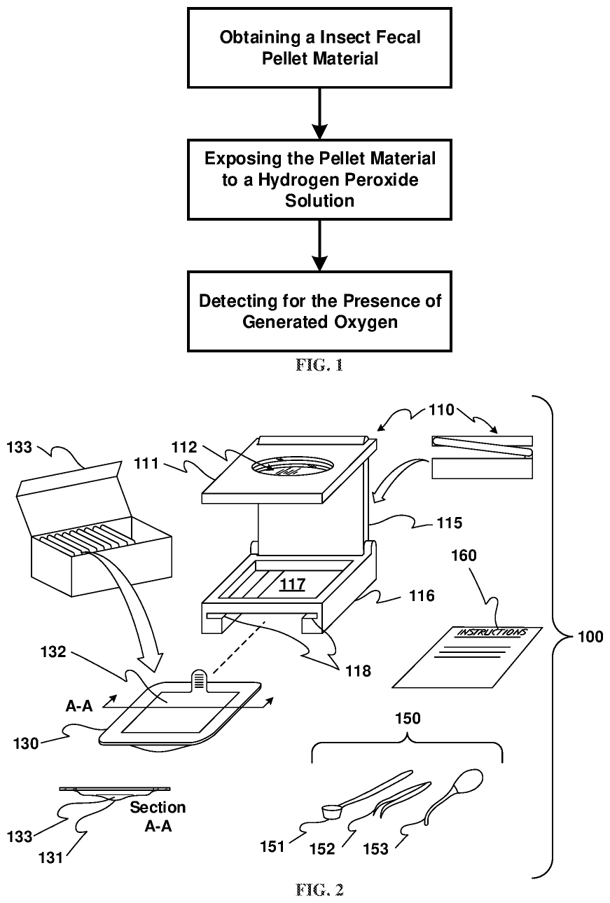

Some embodiments describe a method for characterizing the age of an insect fecal pellet, where the method can comprise: (1) obtaining insect fecal pellet material to be tested; (2) exposing the pellet material to an H₂O₂ solution; and (3) detecting the presence of generated oxygen, where the amount of oxygen generated is inversely proportional to the age of the insect pellet. A non-limiting example of such a method is depicted in FIG. 1. While not wanting to be limited by theory, it is suspected that the presence of living microorganisms (e.g., endosymbionts) in the insects' digestive tract are catalase-positive and their catalase is present in the insects' fresh fecal pellets. Furthermore, it was thought that the pellets as a by-product of insects, contain catalase, an enzyme found in nearly all living organisms exposed to oxygen which reacts with H₂O₂ to create water and oxygen. The inventors believe that the amount of catalase within the excreted pellet decreases over time and the presence of catalase within the pellet can be used as a reliable indicator of pellet age after excretion.

### Obtaining an Insect Pellet

In some embodiments, the step of obtaining insect pellet material can comprise obtaining one or more insect fecal pellets to be tested. In some embodiments, obtaining pellet material can be further dissected into smaller pieces. In some embodiments, obtaining insect pellet material can comprise obtaining at least about 0.001 g, about 0.01 g, about 0.1 g, about 0.5 g, or about 1 g of pellet material for testing, such as about 0.1 g, about 0.5 g, or about 1 g. In some methods, the insect pellet material can comprise pellets excreted from termites (e.g., drywood termites, damp wood termites), wood boring beetles (e.g., bark beetles, powderpost beetles), carpenter ants, ants, cockroach, or wood borers. Some methods, the insect pellets can comprise drywood termite pellets.

Exposing the Pellet Material to an H₂O₂ Solution

In some embodiments, the step of exposing the pellet material to an H₂O₂ solution can comprise placing the pellet material in physical communication with the H₂O₂ solution. In some embodiments, exposing the pellet material to H₂O₂ solution can be by means known in the art such as applying the solution to the pellet material, immersing the pellet material, submerging the pellet material, and the like. In some embodiments, the step exposing the pellet material to H₂O₂ solution can comprise submerging the pellet in about 10 μL to about 10 mL of H₂O₂, such as about 10 μL or about 500 μL or about 3 mL. In some embodiments, the H₂O₂ solution can comprise an aqueous solution where the concentration of H₂O₂ can be between about 0.1 vol. % to about 10 vol. %, about 0.5 vol. % to about 8 vol. %, about 1 vol. % to about 5 vol. %, such as about 3 vol. %. In some methods, the step exposing the pellet material to H₂O₂ solution can comprise submerging the pellet to 3 vol. % H₂O₂ aq. solution.

### Detecting the Presence of Oxygen

In some methods, the detecting the presence of generated oxygen can be done by measuring the for the presence of oxygen. In some embodiments, the measuring for the presence of oxygen can be done by visually detecting bubbles. In some embodiments, the detecting the presence of oxygen can be by detecting the presence of floating pellets. The presence of oxygen whether a pellet is floating or whether it remains submerged, where a floating pellet can be held buoyant by the presence of oxygen gas generated on the surface of the pellet.

In some methods, the step of detecting a colorimetric reaction, where the H₂O₂ solution additionally comprises a colorimetric oxygen sensor compound or dye. The presence of generated oxygen can trigger reactions where one or more colorimetric oxygen sensor compounds or dyes have been added to the H₂O₂ solution. Examples of colorimetric oxygen sensor compounds include but are not limited to: leuco indigo; 2,6-dichlorophenolindophenol (2,6-DCPIP) in the presence of fructose and an organic base; methylene blue (leuco form), TiO₂ and triethanolamine in hydroxyethyl cellulose; indigo carmine solution in its leuco form by a strong reducing agent; and copper or other related copper compounds that form hydrated copper carbonate under the presence of oxygen, water, and CO₂, by forming hydrated copper carbonate. These compounds can change color to aid in the detection of the presence of oxygen and can aid in quick determination of the presence of oxygen in the H₂O₂ solution.

While not wanting to be limited by theory, catalase-positive or recently excreted samples will show obvious color change, oxygen bubble production, or a combination thereof. In contrast, catalase-negative or older samples will not show any obvious color change or oxygen bubble production.

### Type 1 Kit for Detecting the Presence of Active Insects

Some embodiments describe a test kit that can be used to detect the age of insect pellets. Some insects that may be detected by the kits can comprise termites (e.g., drywood termites, damp wood termites), wood boring beetles (e.g., bark beetles, powderpost beetles), carpenter ants, ants, cockroach, or wood borers. Some kits detect drywood termites. Some kits can comprise one or more reaction cartridges and a magnifying device. In some embodiments, the kit can further comprise a pellet handling mechanism, such as forceps, a spatula, or aspirator. Some kits further comprise a light source for illuminating the reaction cartridge. In some kit embodiments, the kit further comprises instructions for using the kit.

In some test kits, the one or more reaction cartridges can comprise a plenum for containing the solution comprising a H₂O₂ solution and the cartridge defines an orifice at the top of the cartridge for the addition of pellet material. The plenum can be configured so that it contains H₂O₂ solution in the center of the plenum, such as a small well, basin, indentation, or depression. For some cartridges, the size of the plenum can be such that at least about 50 μL of solution comprising H₂O₂ can be stored in the cartridge to handle the varying amounts of pellets to be tested. In some kits, the size of the plenum is such that about from 50 μL, about 100 μL, about 200 μL, about 250 μL, about 300 μL, about 400 μL, about 500 μL, about 600 μL, about 700 μL, about 750 μL, about 800 μL, about 900 μL, about 1 mL, about 2 mL, about 2.5 mL, about 3 mL, about 4 mL, about 5 mL, about 6 mL, about 7 mL, about 7.5 mL, about 8 mL, about 9 mL, to about 10 mL, or any combination thereof, of solution comprising H₂O₂ can be stored in the cartridge. In some embodiments, the cartridge orifice can be sealed with a removable seal for storage, where the seal can be removed to expose the plenum for addition and examination of the samples. The seal can be removeable such that it can be opened without specific tools in the field, e.g., a seal with a pull tab.

In some embodiments, the cartridge can be pre-loaded with H₂O₂ solution, such that when a sample is to be tested the seal is removed and the plenum containing the H₂O₂ solution is exposed for testing. In order to preserve the H₂O₂ during storage the H₂O₂ solution should be protected from direct sunlight. For some kits, a box may be used to store multiple cartridges for future use so that they are not exposed to direct sunlight or varying temperatures until use. Multiple reaction cartridges can be included in a kit so that the end users can conduct several tests with the insect fecal pellet samples collected from different locations. In some kits, the H₂O₂ solution can be adjusted by adding an aqueous acid solution so that it is slightly acidic in order to preserve the H₂O₂ solution. In some embodiments, the aqueous acid solution can comprise HCl.

In some kits, the reaction cartridge can further comprise colorimetric oxygen sensor compounds or dyes. In some embodiments, the dyes can be mixed with the H₂O₂ solution. Examples of colorimetric oxygen sensor compounds include but are not limited to: leuco indigo; 2,6-dichlorophenolindophenol (2,6-DCPIP) in the presence of fructose and an organic base; methylene blue (leuco form), TiO₂ and triethanolamine in hydroxyethyl cellulose; indigo carmine solution in its leuco form by a strong reducing agent; and copper or other related copper compounds that form hydrated copper carbonate under the presence of oxygen, water, and CO₂, by forming hydrated copper carbonate. These compounds can change color to aid in the detection of the presence of oxygen and can aid in quick determination of the presence of oxygen in the H₂O₂ solution.

In some embodiments, the reaction cartridges can be at least translucent so that light, natural or generated, can illuminate the H₂O₂ solution within the plenum for observation. In some embodiments, the reaction cartridges can be or transparent. In some kits, the cartridge is not transparent or translucent but rather the plenum is illuminated via the opening after the seal is removed. For some kits, the color of the material of the reaction cartridge can be chosen to protect against degradation of the H₂O₂ solution. Some reaction cartridges can comprise silica, or plastic that can safely store hydrogen peroxide. In some embodiments, the reaction cartridges can comprise borosilicate glass, polypropylene, polycarbonate, polyphenylene sulfide, polyethylene, high-density polyethylene, polyvinyl chloride, chlorinated polyvinyl chloride, polytetrafluoroethylene, polyvinylidene fluoride, co-polymers of vinylidene fluoride, or hexafluoropropylene. In some embodiments, the removable seal can comprise aluminum, Al—Mg alloys, polypropylene, polycarbonate, polyphenylene sulfide, polyethylene, high-density polyethylene, polyvinyl chloride, chlorinated polyvinyl chloride, polytetrafluoroethylene, polyvinylidene fluoride, co-polymers of vinylidene fluoride, or hexafluoropropylene. Materials should be chosen to maintain the stability of the H₂O₂ when the seal is unbroken, and the cartridges are stored in a cool, dark environment. It is known that the rate of its decay of H₂O₂ is influenced by the solution concentration, the storage temperature, and the solution pH. But if properly stored, it is known that 3% H₂O₂ solution is commonly used as a disinfectant typically has a shelf life of at least a year and up to three years if the bottle is unopened.

In some kit embodiments, the kits comprise a magnification device. In some kits the magnification device can be portable. In some kits, the magnification device can be folded for storage so that it can easily be stored, carried, or used in field conditions. In some magnification devices, the device comprises a lens, a lens holder defining a cavity in which the lens is physically secured by the lens holder within the cavity, a base for holding the reaction cartridge, and a linkage for holding the lens holder a desired distance from the reaction cartridge. In some embodiments, the base can define a measurement cavity, the measurement cavity extending from the upper portion of the base to where the reaction cartridge is held, such that the reaction cartridge plenum and orifice, when inserted in the base are all in optical communication with the lens, e.g., can be clearly seen from the top of the base. In some embodiments the base, can define a slot where the reaction cartridge can be inserted into the base and held in place for measurements, similar to a microscope stage. In some embodiments, the reaction cartridge can be inserted into the side of the base. In some embodiments, the reaction cartridge can be inserted by placing it into the base from the top, through the measurement cavity. In some embodiments, the reaction cartridge can be inserted through the bottom of the base.

In some embodiments, the linkage can comprise a track whereby the attachment points of the lens holder can slide along the length of the linkage within the track such that the distance of the lens from the reaction cartridge can be adjusted by moving the lens holder with respect to the base.

For some test kits, the kit can comprise a light source. The light sources can to facilitate the inspection of the generated oxygen by the insect fecal pellets by illuminating the reaction cartridge plenum. The light source can comprise an incandescent light bulb, or one or more light emitting diodes. In some embodiments the light source can be mounted on a separate light plate. In some kits, the magnification device can physically rest on the light plate such that light is shown through the reaction cartridge from below. In some kits, the light source can be mounted on the linkage of the magnification device such that the light source illuminates the reaction cartridge from above shining on the H₂O₂ solution through the cartridge orifice. Some light sources can be internally powered, such as by a battery. Some light sources can be externally powered, via a power plug.

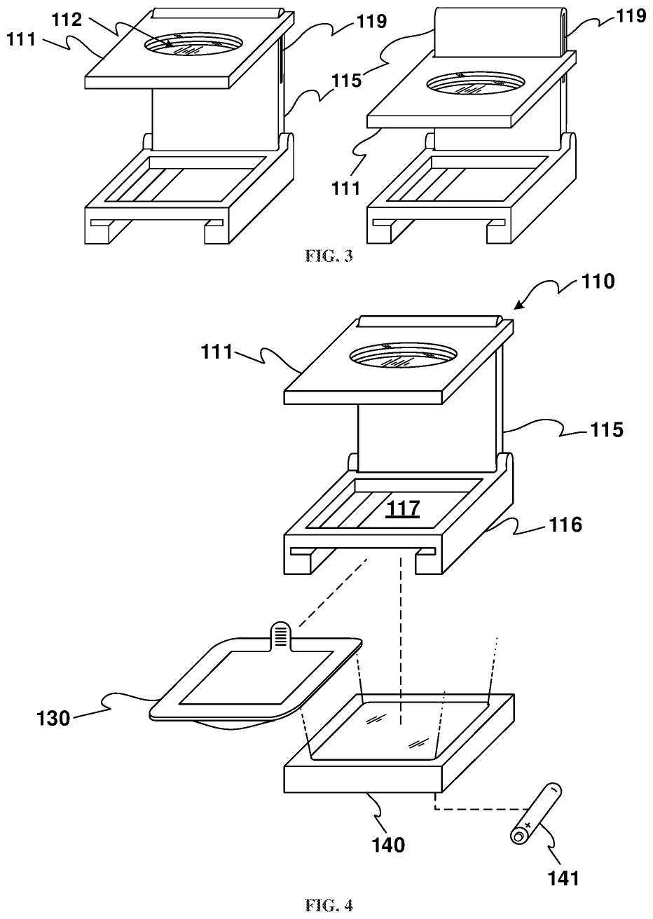

A non-limiting example of a test kit embodiment is shown in FIG. 2. The kit, 100, comprises a magnification device, 110, and a reaction cartridge, 130. The magnification device can comprise a lens holder, 111, with a lens, 112, a linkage, 115, for supporting the lens holder to the base, 116.

The reaction cartridge, 130, can contain a plenum, 131, such that the solution comprising H₂O₂, 133, can be held in the cartridge. The reaction cartridge can have a removable seal, 132, which is removed before use. Some kit may also contain an optional box, 133, for storing the multiple reaction cartridges for future uses.

The base, 116, can define a measurement cavity, 117, where the reaction cartridge, 130, can be inserted into the base for use and measurements. The reaction cartridge, 130, can be placed such that when the seal, 132, is removed, the solution comprising H₂O₂, 133, can be in optical communication with the lens, 112. In some embodiments, the base can define a slot, 118, where the reaction cartridge, 130, can be held in place during measurements.

In some kits, a non-limiting example presented in FIG. 3, the linkage, 115, can also comprise a track, 119, such that the distance between the lens holder and the base can be adjusted.

In some kit embodiments, ambient light can provide the light source for illuminating the reaction cartridge. Some kits can further comprise one or more light sources, such as a light plate or a lamp. In some embodiments, the light source can comprise, LEDs, light bulbs, compact fluorescents, or the like. In some kits, as depicted in FIG. 4, can further comprise a light plate, 140, which can illuminate the reaction cartridge, 130, from underneath. In some embodiments, the light plate can be externally powered, not shown. In some embodiments, the light plate can be battery, 141, powered.

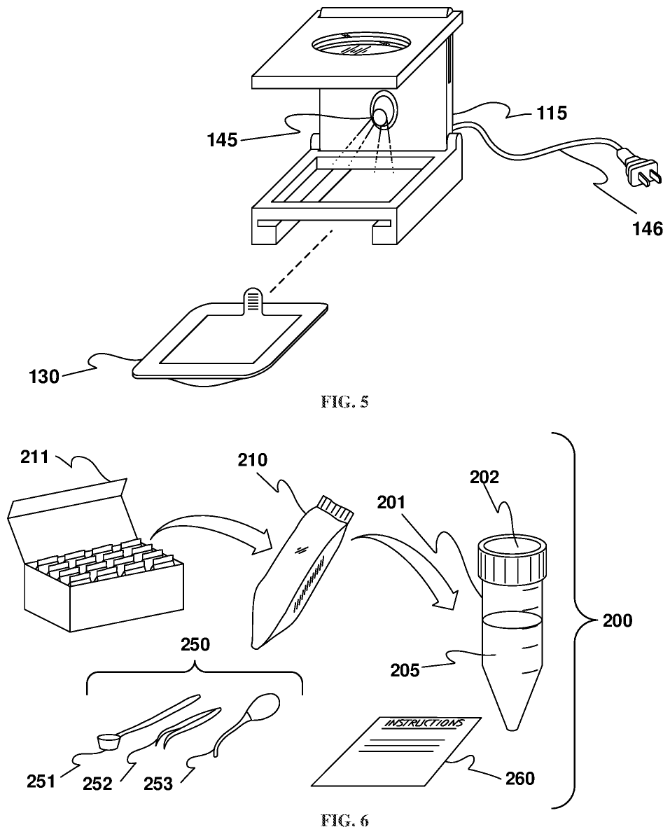

In some kits, a non-limited example shown in FIG. 5, a lamp, 145, can be mounted on the linkage, 115, such that the reaction cartridge, 130, is illuminated from above. In some kit embodiments, the lamp, 145, can be powered by a battery, not shown, contained within the linkage. In some embodiments, the lamp, 145, can be externally powered, such as through an electrical interface, 146.

As shown in FIG. 2, some kits can further comprise one or more tools for transferring the fecal pellets for testing, 150. The tools for transferring of the pellets can comprise a small scoop, 151, forceps, 152, or an aspirator, 153. In some embodiments, the scoop can have a capacity from about 10 μL to about 50 μL. In some embodiments, the scoop can be shaped such that it can pick up pellets from a flat surface, e.g., a spatula.

Some kits can also include instructions, 160, for using the kit, as shown in FIG. 2. In some kits, the instructions can outline the one or more of the aforedescribed methods for detecting the age of insect excrement. Some instructions can comprise the steps of:
(1) obtaining insect pellet material to be tested;
(2) exposing the pellet material to an H₂O₂ solution; and
(3) detecting the presence of generated oxygen,
   where the amount of oxygen is inversely proportional to the age of the insect pellet. In some kits, the instructions can direct the use tools for transferring to transfer the pellet from its discovery location to the reaction cartridge containing the H₂O₂ solution. In some instructions, the step of exposing the pellet to H₂O₂ solution can comprise placing the pellet in H₂O₂ gently into the reaction cartridge to reduce entrainment of oxygen with the pellet into the H₂O₂. Some instructions can direct the detection the presence of oxygen by determining of bubbles are present, pellets are floating, or of the appropriate colorimetric reaction occurs if colorimetric oxygen sensor compounds or dyes are included in the kit. In some kit instructions, the step of obtaining insect fecal pellet material comprises obtaining pellets excreted from a termite, wood boring beetle, carpenter ant, ant, cockroach, or wood borer. In some kit instructions, the step of obtaining insect fecal pellet material comprises obtaining pellets excreted from a drywood termite.

A Type I test kit design may be suited for scenarios with limited amounts of fecal pellets available for testing, such as 0.1 g or less. The test kit includes the following components.

### Type 2 Kit for Detecting the Presence of Active Insects

Some embodiments describe a second type of test kit to detect the age of insect pellets. Some insects that may be detected by the kits can comprise termites (e.g., drywood termites, damp wood termites), wood boring beetles (e.g., bark beetles, powderpost beetles), carpenter ants, ants, cockroach, or wood borers. Some kits detect drywood termites. In some embodiments, the kit can comprise one or more reaction tubes and H₂O₂ solution within the tubes.

In some embodiments, the reaction tube can be a cylindrical tube that has a closed end at the bottom and defines an orifice on at the top. In some tubes, the closed end can taper to a smaller cross section to concentrate the test material, e.g. a conical shape. Some kits can further comprise a removable cap, where the orifice can be sealed by the cap. Some tubes can have a capacity of from about 500 μL, about 1 mL, about 3 mL, about 5 mL, about 6 mL, about 10 mL, about 15 mL, about 25 mL, to about 50 mL, or any combination thereof, such as about 500 μL or about 3 mL. In some kits, a fraction of the internal volume of the reaction tube can be filled with a solution comprising H₂O₂. In some kits, the H₂O₂ solution can be adjusted by adding an aqueous acid solution so that it is slightly acidic in order to preserve the H₂O₂ solution. In some embodiments, the aqueous acid solution can comprise HCl.

In some kits, the reaction tube can further comprise colorimetric oxygen sensor compounds or dyes mixed with the H₂O₂ solution. Examples of colorimetric oxygen sensor compounds include but are not limited to: leuco indigo; 2,6-dichlorophenolindophenol (2,6-DCPIP) in the presence of fructose and an organic base; methylene blue (leuco form), TiO₂ and triethanolamine in hydroxyethyl cellulose; indigo carmine solution in its leuco form by a strong reducing agent; and copper or other related copper compounds that form hydrated copper carbonate under the presence of oxygen, water, and CO₂, by forming hydrated copper carbonate. These compounds can change color to aid in the detection of the presence of oxygen and can aid in quick determination of the presence of oxygen in the H₂O₂ solution.

In order to preserve the H₂O₂ during storage the H₂O₂ solution should be protected from direct sunlight. In some kits, the tubes can be individually stored in removable packaging to protect them from direct sunlight. For some kits, a box may be used to store multiple cartridges for future use so that they are not exposed to direct sunlight or varying temperatures until use. Multiple reaction cartridges can be included in a kit so that the end users can conduct several tests with the insect fecal pellet samples collected from different locations.

In some kits the reaction tube can be disposable. For some kits, the reaction tubes may be reusable. Some reaction tubes can be transparent so that the catalase test reaction can be readily detected. In some embodiments, the reaction tube can comprise borosilicate glass, polypropylene, polycarbonate, polyphenylene sulfide, polyethylene, high-density polyethylene, polyvinyl chloride, chlorinated polyvinyl chloride, polytetrafluoroethylene, polyvinylidene fluoride, co-polymers of vinylidene fluoride, or hexafluoropropylene.

For some embodiments that at least about 500 μL of H₂O₂ solution is included in the tube to be able to accommodate varying amounts of insect pellets that will be introduced into the reaction tube. In some embodiments, about 500 μL to about 3 mL of H₂O₂ solution can be included in the reaction tube.

A non-limiting example of a test kit embodiment is shown in FIG. 6. The kit, 200, comprises a reaction tube, 201, with a removable cap, 202, containing a solution comprising H₂O₂, 205. In some kits, the tube can be sealed in a removable packaging, 210. In some kits, a plurality of reaction tubes can be provided, the tube stored in a box, 211.

As shown in FIG. 6, some kits can further comprise one or more tools for transferring the fecal pellets for testing, 250. The tools for transferring of the pellets can comprise a small scoop, 251, forceps, 252, or an aspirator, 253. In some embodiments, the scoop can have a capacity from about 10 μL to about 50 μL. In some embodiments, the scoop can be shaped such that it can pick up pellets from a flat surface, e.g., a spatula.

Some kits can also include instructions, 260, for using the kit, as shown in FIG. 6. In some kits, the instructions can outline the one or more of the aforedescribed methods for detecting the age of insect excrement. Some instructions can comprise the steps of:
(1) obtaining insect pellet material to be tested;
(2) exposing the pellet material to an H₂O₂ solution; and
(3) detecting the presence of generated oxygen,
   where the amount of oxygen is inversely proportional to the age of the insect pellet. In some kits, the instructions can direct the use tools for transferring to transfer the pellet from its discovery location to the reaction cartridge containing the H₂O₂ solution. In some instructions, the step of exposing the pellet to H₂O₂ solution can comprise placing the pellet in H₂O₂ gently into the reaction tube to reduce entrainment of oxygen with the pellet into the H₂O₂. Some instructions can direct the detection the presence of oxygen by determining of bubbles are present, pellets are floating, or of the appropriate colorimetric reaction occurs if colorimetric oxygen sensor compounds or dyes are included in the kit. In some kit instructions, the step of obtaining insect fecal pellet material comprises obtaining pellets excreted from a termite, wood boring beetle, carpenter ant, ant, cockroach, or wood borer. In some kit instructions, the step of obtaining insect fecal pellet material comprises obtaining pellets excreted from a drywood termite.

### EMBODIMENTS

The following embodiments are specifically contemplated by this disclosure:
Embodiment 1. A method for characterizing the age of an insect fecal pellet, the method comprising: (1) obtaining insect fecal pellet material to be tested; (2) exposing the pellet material to an H₂O₂ solution; and (3) detecting the presence of generated oxygen, where the amount of oxygen generated is inversely proportional to the age of the insect pellet.
Embodiment 2. The method of Embodiment 1, where the step of obtaining insect fecal pellet material comprises obtaining at least about 0.001 g of fecal pellet material.
Embodiment 3. The method of Embodiment 1 or 2, where the step of obtaining insect fecal pellet material comprises obtaining pellets excreted from a termite, a wood boring beetle, a carpenter ant, an ant, a cockroach, or a wood borer.
Embodiment 4. The method of Embodiment 1, 2, or 3, where the step of obtaining insect fecal pellet material comprises obtaining pellets excreted from a drywood termite.
Embodiment 5. The method of Embodiment 1, 2, 3, or 4, where the step of exposing the pellet material to an H₂O₂ solution comprises exposing the pellet material to an aqueous solution of 0.1 vol. % to 10 vol. % H₂O₂.
Embodiment 6. The method of Embodiment 1, 2, 3, 4, or 5, where the step of exposing the pellet material to an H₂O₂ solution comprises placing the pellet material in physical communication with the H₂O₂ solution.
Embodiment 7. The method of Embodiment 1, 2, 3, 4, 5, or 6, where the step of exposing comprises applying the solution to the pellet material, immersing the pellet material, or submerging the pellet material.
Embodiment 8. The method of Embodiment 1, 2, 3, 4, 5, 6, or 7, where the step of detecting the presence of generated oxygen is by detecting bubbles.
Embodiment 9. The method of Embodiment 1, 2, 3, 4, 5, 6, 7, or 8, where the step of detecting the presence of generated oxygen is by detecting floating pellets.
Embodiment 10. The method of Embodiment 1, 2, 3, 4, 5, 6, 7, 8, or 9, where the step of detecting the presence of generated oxygen is by detecting a colorimetric reaction, where the H₂O₂ solution additionally comprises colorimetric oxygen sensor compounds or dyes.
Embodiment 11. A kit for detecting the age of an insect fecal pellet, the kit comprising: one or more reaction cartridges, where the reaction cartridges can comprise a plenum for containing at least 50 μL of solution comprising a H₂O₂ solution and the cartridge defines an orifice at the top of the cartridge for the addition of pellet material; a magnifying device; and a set of instructions, the instructions comprising the steps of: (1) obtaining insect pellet material to be tested, (2) exposing the pellet material to an H₂O₂ solution, and (3) detecting the presence of generated oxygen.
Embodiment 12. The kit of Embodiment 11, where the reaction cartridges are at least translucent so that light, natural or generated, can illuminate the H₂O₂ solution.
Embodiment 13. The kit of Embodiment 11 or 12, where the reaction cartridges further comprise a removable seal for sealing the cartridge orifice, which is removed to expose the plenum for addition and examination of the samples.
Embodiment 14. The kit of Embodiment 11, 12, or 13, where the reaction cartridge can further comprise colorimetric oxygen sensor compounds or dyes mixed in with the H₂O₂ solution.
Embodiment 15. The kit of Embodiment 11, 12, 13, or 14, where the magnification device comprises a lens, a lens holder defining a cavity in which the lens is physically secured by the lens holder within the cavity, a base for holding the reaction cartridge, the base defining a measurement cavity, the measurement cavity extending from the upper portion of the base to where the reaction cartridge is held, such that the reaction cartridge plenum and orifice, when inserted in the base are all in optical communication with the lens, and a linkage for holding the lens holder a desired distance from the reaction cartridge.
Embodiment 16. The kit of Embodiment 11, 12, 13, 14, or 15, where the linkage comprises a track whereby the attachment points of the lens holder can slide along the length of the linkage within the track such that the distance of the lens from the reaction cartridge are adjustable.
Embodiment 17. The kit of Embodiment 11, 12, 13, 14, 15, or 16, further comprising a light source for illuminating the reaction cartridge plenum.
Embodiment 18. The kit of Embodiment 11, 12, 13, 14, 15, 16, or 17, further comprising one or more tools for transferring the fecal pellets for testing selected from the group consisting a small scoop, forceps, or an aspirator.
Embodiment 19. The kit of Embodiment 11, 12, 13, 14, 15, 16, 17, or 18, where the instruction step of obtaining insect fecal pellet material to be tested comprises obtaining pellets excreted from a termite, a wood boring beetle, a carpenter ant, an ant, a cockroach, or a wood borer.
Embodiment 20. The kit of Embodiment 11, 12, 13, 14, 15, 16, 17, 18, or 19, where the instruction step of obtaining insect fecal pellet material to be tested comprises obtaining pellets excreted from a drywood termite.
Embodiment 21. A kit for detecting the age of an insect fecal pellet, the kit comprising: one or more reaction tubes, the reaction tube having a capacity of 500 μL to 50 mL and a closed end at the bottom, defining an orifice at the top; a removable cap, where the tube orifice can be sealed by the cap; an H₂O₂ solution, where a fraction of the internal volume of the one or more tubes is filled with the solution; and a set of instructions, the instructions comprising the steps of: (1) obtaining insect pellet material to be tested, (2) exposing the pellet material to an H₂O₂ solution, and (3) detecting the presence of generated oxygen.
Embodiment 22. The kit of Embodiment 21, where the one or more reaction tubes are individually stored in removable packaging to protect them from direct sunlight.
Embodiment 23. The kit of Embodiment 21 or 22, the reaction tube further comprising colorimetric oxygen sensor compounds or dyes mixed in with the H₂O₂ solution.
Embodiment 24. The kit of Embodiment 21, 22, or 23, further comprising one or more tools for transferring the fecal pellets for testing selected from the group consisting a small scoop, forceps, or an aspirator.
Embodiment 25. The kit of Embodiment 21, 22, 23, or 24, where the instruction step of obtaining insect fecal pellet material to be tested comprises obtaining pellets excreted from a termite, a wood boring beetle, a carpenter ant, an ant, a cockroach, or a wood borer.
Embodiment 26. The kit of Embodiment 21, 22, 23, 24, or 25, where the instruction step of obtaining insect fecal pellet material to be tested comprises obtaining pellets excreted from a drywood termite.

### EXAMPLES

It has been discovered that the composite embodiments described herein may provide the ability to facilitate delocalization of stress at the point of impact, thereby reducing the probability of catastrophic failure of the underlying structure. These benefits are further demonstrated by the following example, which are intended to be illustrative of the embodiments of the disclosure but are not intended to limit the scope or underlying principles in any way.

### Example 1: Verification of Method for Detecting Termite Pellet Age—Long Time Frame

For the examination, a small amount of termite fecal pellets was dropped into a about 10 μL of H₂O₂ (3% aq. sol., Sigma Aldrich, St. Louis, MO, USA). The pellets tested were fresh pellets between 1 day and a few weeks since excrement, and old fecal pellets approximately 25 years old. Then the pellets in solution were then observed under high magnification to detect for the presence of oxygen bubbles.

It was observed, as shown in Table 1, that the fresh pellets generated oxygen bubbles but that the old pellets did not generate oxygen bubble. The result is that the method by detecting oxygen bubbles can detect the age of a termite pellet.

TABLE 1
Comparison of Presence of Gas Generate Versus Age of Pellets.

| | Presence of Gas |
|---|---|
| Age of Pellets Tested | (Solid - Yes) |
| New Fecal Pellets (1 day-weeks old) | ● |
| Old Fecal Pellets (25 years old) | ○ |

### Example 2: Verification of Method for Detecting Termite Pellet Age—Short Time Frame

For this example, a controlled environment was used to validate the specific response of the method to varying termite pellet ages. Western drywood termites (Incisitermes minor) were collected on Dec. 13, 2018. The termites were then kept in three separate petri dishes and were provided with small shelters made of balsa wood panels. The termites consumed the balsa wood panels and produced fecal pellets over a period of a month. On Jan. 14, 2019, three batches of freshly produced fecal pellets were then collected from these termites and kept at 26° C. in separate petri dishes without access to the live termite colonies. Thirty fecal pellets were randomly collected from each of the petri dishes and subsequently examined for their reaction with the hydrogen peroxide solution.

The results are shown in Table 2 provide the method's granular ability to quantitively demonstrate age of a termite pellet. It was observed that on Day 1 after collection, an average of 87.8% of pellets produced oxygen bubbles as they react with hydrogen peroxide. On Day 30 after collection, an average 21.1% of fecal pellets produced bubbles as they react with hydrogen peroxide. On Day 60 after collection, an average 4.4% of fecal pellets produced bubbles as they react with hydrogen peroxide. On Day 135 after collection, none of the sampled fecal pellets produced bubble as they were mixed with hydrogen peroxide. These results show that the method provides an accurate measure of the age of a termite pellet, even within a short period of time. Such a test can be utilized to indicate whether there is an active infestation of insects.

TABLE 2
Comparison of Percentage of Pellets that Generated Gas Versus Age.

| Tested Pellets' Age After | Percentage Reacting to |
|---|---|
| Collection | Produce Oxygen [%] |
| 1 Day Old Fecal Pellets | 87.8 |
| 30-Day Old Fecal Pellets | 21.1 |
| 60-Day Old Fecal Pellets | 4.4 |
| 135-Day Old Fecal Pellets | 0 |

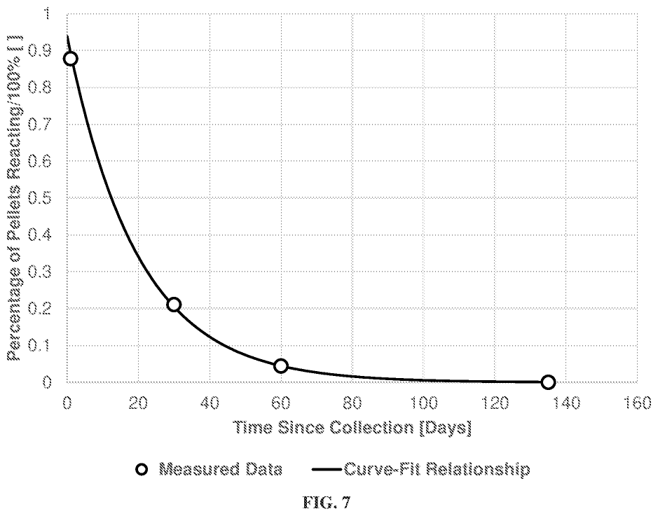

As shown in FIG. 7, curve fitting the percentage of pellets reacting to product oxygen produces the approximate relationship between age after collection and the % reacting:

$$\frac{Pellets Reacting \%}{100 \%} = {e}^{( 0.0507 \cdot T - 0.0639 )}$$

where T is the time since the that the pellets were collected, where T is 135 days or less. While this relationship is for number of pellets reacting, a similar relationship can be done for amount of gas generated or magnitude of change of a colorimetric detector. The data indicates that the methods herein can be quantitatively used to determine the age of the pellet with the potential for day-level accuracy.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed considering the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice ant of the embodiments disclosed in the present disclosure.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is to be understood that the embodiments of the invention disclosed herein are illustrative of the principles of the present invention. It should be understood that the disclosed subject matter is in no way limited to a particular methodology, protocol, and/or reagent, etc., as described herein. Various modifications or changes to or alternative configurations of the disclosed subject matter can be made in accordance with the teachings herein without departing from the spirit of the present specification. Accordingly, the present invention is not limited to that precisely as shown and described.

What is claimed is:

1. A method for characterizing the age of an insect fecal pellet, the method comprising: (1) obtaining insect fecal pellet material to be tested; (2) exposing the pellet material to an H₂O₂ solution; and (3) detecting the presence of generated oxygen, where the amount of oxygen generated is inversely proportional to the age of the insect pellet.

2. The method of claim 1, where the step of obtaining insect fecal pellet material comprises obtaining at least about 0.001 g of fecal pellet material.

3. The method of claim 1, where the step of obtaining insect fecal pellet material comprises obtaining pellets excreted from a termite, a wood boring beetle, a carpenter ant, an ant, a cockroach, or a wood borer.

4. The method of claim 2, where the step of obtaining insect fecal pellet material comprises obtaining pellets excreted from a drywood termite.

5. The method of claim 1, where the step of exposing the pellet material to an H₂O₂ solution comprises exposing the pellet material to an aqueous solution of 0.1 vol. % to 10 vol. % H₂O₂.

6. The method of claim 1, where the step of exposing the pellet material to an H₂O₂ solution comprises placing the pellet material in physical communication with the H₂O₂ solution.

7. The method of claim 6, where the step of exposing comprises applying the solution to the pellet material, immersing the pellet material, or submerging the pellet material.

8. The method of claim 1, where the step of detecting the presence of generated oxygen is by detecting bubbles.

9. The method of claim 1, where the step of detecting the presence of generated oxygen is by detecting floating pellets.

10. The method of claim 1, where the step of detecting the presence of generated oxygen is by detecting a colorimetric reaction, where the H₂O₂ solution additionally comprises colorimetric oxygen sensor compounds or dyes.

11. A kit for detecting the age of an insect fecal pellet, the kit comprising: one or more reaction cartridges, where the reaction cartridges can comprise a plenum for containing at least 50 μL of solution comprising a H₂O₂ solution and the cartridge defines an orifice at the top of the cartridge for the addition of pellet material; a magnifying device; and a set of instructions, the instructions comprising the steps of: (1) obtaining insect pellet material to be tested, (2) exposing the pellet material to an H₂O₂ solution, and (3) detecting the presence of generated oxygen.

12. The kit of claim 11, where the reaction cartridges are at least translucent so that light, natural or generated, can illuminate the H₂O₂ solution.

13. The kit of claim 11, where the reaction cartridges further comprise a removable seal for sealing the cartridge orifice, which is removed to expose the plenum for addition and examination of the samples.

14. The kit of claim 11, where the reaction cartridge can further comprise colorimetric oxygen sensor compounds or dyes mixed in with the H₂O₂ solution.

15. The kit of claim 11, where the magnification device comprises a lens, a lens holder defining a cavity in which the lens is physically secured by the lens holder within the cavity, a base for holding the reaction cartridge, the base defining a measurement cavity, the measurement cavity extending from the upper portion of the base to where the reaction cartridge is held, such that the reaction cartridge plenum and orifice, when inserted in the base are all in optical communication with the lens, and a linkage for holding the lens holder a desired distance from the reaction cartridge.

16. The kit of claim 11, where the linkage comprises a track whereby the attachment points of the lens holder can slide along the length of the linkage within the track such that the distance of the lens from the reaction cartridge are adjustable.

17. The kit of claim 11, further comprising a light source for illuminating the reaction cartridge plenum.

18. The kit of claim 11, further comprising one or more tools for transferring the fecal pellets for testing selected from the group consisting a small scoop, forceps, or an aspirator.

19. The kit of claim 11, where the instruction step of obtaining insect fecal pellet material to be tested comprises obtaining pellets excreted from a termite, a wood boring beetle, a carpenter ant, an ant, a cockroach, a wood borer, or a drywood termite.

20. A kit for detecting the age of an insect fecal pellet, the kit comprising: one or more reaction tubes, the reaction tube having a capacity of 500 μL to 50 mL and a closed end at the bottom, defining an orifice at the top; a removable cap, where the tube orifice can be sealed by the cap; an H₂O₂ solution, where a fraction of the internal volume of the one or more tubes is filled with the solution; and a set of instructions, the instructions comprising the steps of: (1) obtaining insect pellet material to be tested, (2) exposing the pellet material to an H₂O₂ solution, and (3) detecting the presence of generated oxygen.
